(19) United States
(12) Patent Application Publication
(10) Pub. No.: US 2008/0255524 A1
(43) Pub. Date: Oct. 16, 2008

(54) Structure of the syringe cover including the barrel

(75) Inventor: Meng-Shih Fu, Taipei (TW)

Correspondence Address: BACON & THOMAS, PLLC, 625 SLATERS LANE, FOURTH FLOOR, ALEXANDRIA, VA, 22314-1176, US

(73) Assignee: Vantex Biotechnology Co., Ltd., Tortola (VG)

(21) Appl. No.: 11/783,628

(22) Filed: Apr. 11, 2007

(51) Int. Cl. A61M 5/32

(52) U.S. Cl. 604/198

(57) ABSTRACT

A structure of the syringe cover including the barrel is a design that the aperture end of the hollow barrel of a safety syringe is coupled to a syringe cover. While the plunger is pulled back, the needle of the safety syringe disengages from the plunger, and the needle and the needle holder integral are received within the hollow barrel. With the syringe cover coupled to the aperture end of the hollow barrel, the medical personnel are prevented from being pricked with needles because of exposure of needles retained within the hollow barrels by mutual jostle between syringes in medical wastes.

### BACKGROUND OF THE INVENTION

1) Field of the Invention

The present invention relates to a structure of the syringe cover including the barrel, and more particularly to a syringe structure wherein the aperture end of the barrel having the identical diameter to the syringe cover is coupled to the syringe cover, and utilized to prevent exposure of needles owing to mutual jostle of used syringes, and implemented in a structure of the syringe with a capacity from 0.3 cc to 60 cc.

2) Description of the Prior Art

Prior to the present invention, the inventor invented two configurations for a syringe including a self-destruction and a safety syringe, moreover successively acquired patents for same in the United States of America, Taiwan, and China, having patent numbers of M253366, ZL02239125.8, U.S. Pat. No. 6,488,657 B1, U.S. Pat. No. 5,993,419 respectively, objective of which was to provide a traditional syringe with a configuration that only allowed single-usage, and did not allow for recycling after the syringe was discarded, and with such a configuration to realize complete eradication of infection from needles used in medical treatment.

In light of the aforementioned related patents, a primary structural characteristic of the self-destruction syringe is that the needle of the safety syringe disengages from the plunger, and the syringe needle and the needle holder integral are received within the hollow barrel, while the plunger pulled back, thereby achieving functionality of a safety design, wherewith medical personnel are prevented from being pricked by the needle.

However, because of jostle of cotton swabs and other needles within medical wastes, needles retained in the hollow barrels are extruded to prick the skin of medical personnel.

Thus, closing action at the aperture end of the hollow barrel is a cardinal key of the self-destruction syringe or the safety syringe, and therefore structural design at the aperture end is of particular importance.

### SUMMARY OF THE INVENTION

A primary objective of a structure of the syringe cover including the barrel is to provide a syringe cover that can be implemented in the self-destruction and safety syringe, and thereby accommodate syringes of differing capacity. Such a structure allows for implementation in syringes with different capacity of 3 cc, 5 cc and 10 cc, whereby because the outer diameter at the aperture end of the hollow barrel must be the same to the inner diameter of the syringe cover, and the syringe cover can be configured to the end of a barrel, but length of the syringe cover can be designed to meet different lengths of needles in accord with necessary specifications (lengths) of needles, thereby require an extraordinary need for a precisely structural design at the end.

Another objective of a structure of the syringe cover including the barrel, especially for point of use biohazard container or point of use sharp container, is to provide a characteristic that the needle is not exposed after the syringe is used, so that to discard syringes to a specific box for medical wastes is not necessary.

To enable a further understanding of the said objectives and the technological contents of the invention herein, the description of the preferred embodiments with drawings is listed as follows.

### BRIEF DESCRIPTION OF THE DRAWINGS

FIG. 1 shows an exploded elevational view of the structure according to the known syringe.

FIG. 2 shows a drawing of a cross sectional view of containment in a barrel of the syringe according to the known syringe.

FIG. 3 shows an exploded elevational view of the structure of the syringe cover including the barrel according to the present invention.

FIG. 4 shows a drawing of a cross sectional view for the structure of the syringe cover including the barrel in usage according to the present invention.

FIG. 5a shows a first drawing of a cross sectional view for the structure of the syringe cover including the barrel in usage according to the present invention.

FIG. 5b shows a second drawing of a cross sectional view for the structure of the syringe cover including the barrel in usage according to the present invention.

FIG. 6a shows a first drawing for length of the syringe cover of the syringe cover including the barrel in usage according to the present invention.

FIG. 6b shows a second drawing for length of the syringe cover of the syringe cover including the barrel in usage according to the present invention.

### DETAILED DESCRIPTION OF THE PREFERRED EMBODIMENTS

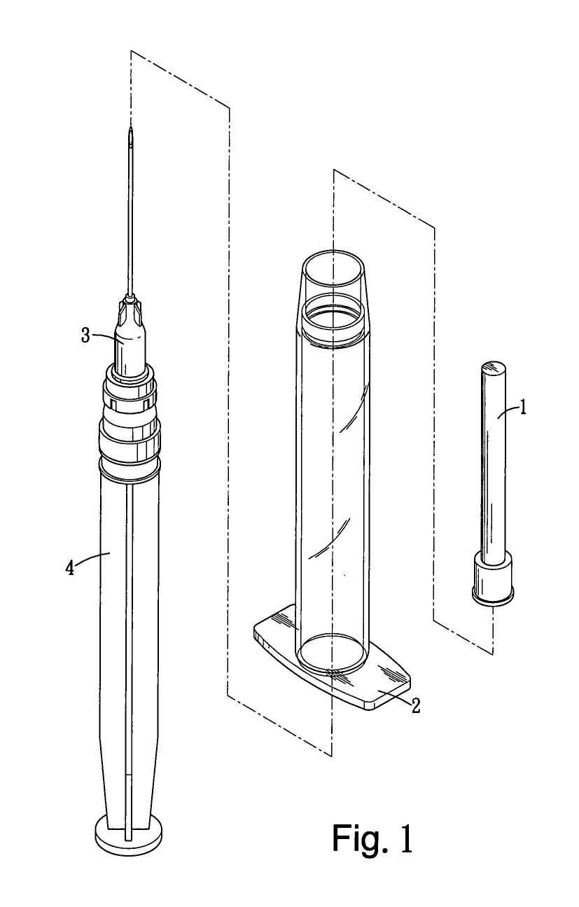

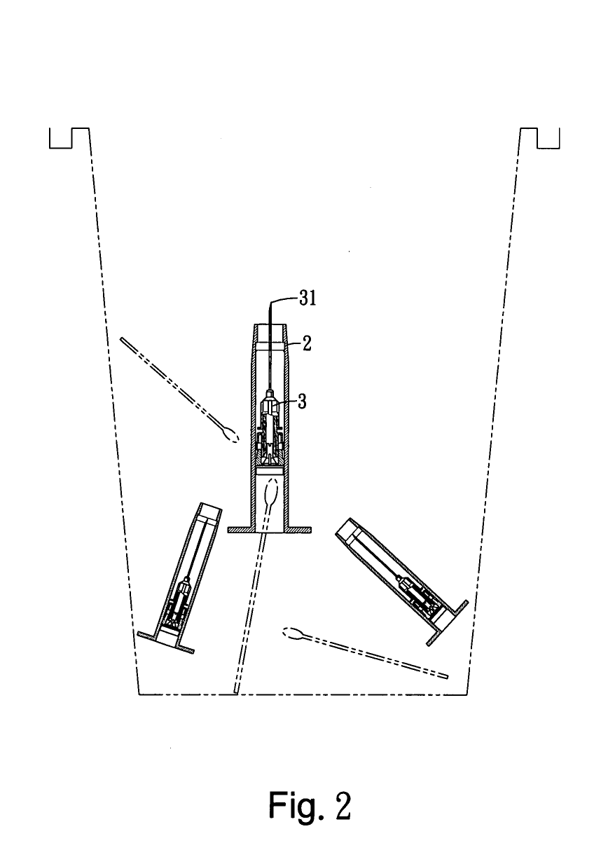

Referring to FIGS. 1 and 2 simultaneously, which show an embodiment of a known self-destruction syringe, wherein structure of the safety syringe is primarily configured to comprise a needle cover 1, a hollow barrel 2, a needle 3, and a plunger 4.

With the needle 3 used, the plunger 4 is pulled back and the needle 3 is received within the hollow barrel 2, whereupon the plunger 4 is broken and separated so as to the needle 3 is preserved within the hollow barrel 2, and thereby prevents medical personnel to be pricked, and thus realizes a safety design.

Those used barrels 2 are collected in bags for destroy later. However, mutual jostle of a great quantity of barrels 2 having various capacities and cylinder diameters causes the pointed end 31 of the needle 3 to extrude outside the hollow barrel 2, and thereby leads to prick on the skin of medical personnel, and thus fails to guarantee personal safety.

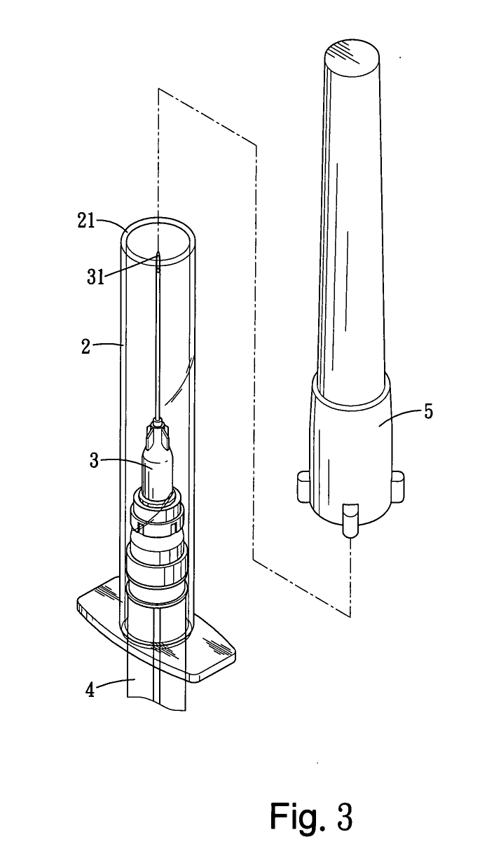

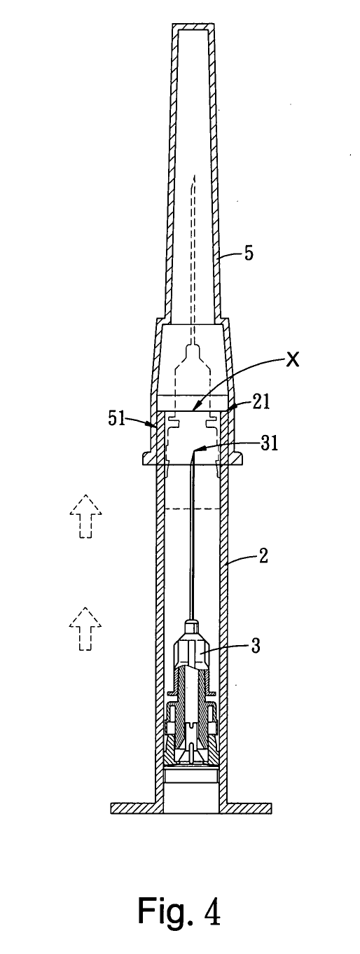

Further, referring FIGS. 3 and 4 simultaneously, which show an embodiment of a structure of the syringe cover including the barrel of the present invention, wherein the structure is primarily configured to comprise a hollow barrel 2, a needle 3, a plunger 4, and a syringe cover 5.

Wherein the syringe cover 5 assumes a conical form, and the inner wall 51 indicates an inclined guide face. The diameter of the inner wall 51 of the syringe cover 5 is adapted to and coupled to the outer diameter of the aperture end 21 of the hollow barrel 2, and thereupon a closing situation is formed at the aperture end of the hollow barrel 2.

With injection and coupling finished by the medical personnel, the plunger 4 is pulled back and disengaged from the needle 3, whereupon the needle 3 is received within the hollow barrel 2. Then, for prevention of injury of pricking medical personnel resulting from exposure of needles at the aperture end 21 of the hollow barrel 2 by mutual jostle between cotton swabs and syringes having various capacities in the medical wastes, the syringe cover 5 is coupled to the end 21 of the hollow barrel 2, so as to become a hermetic sealed design, and then collected to bags for medical wastes. Furthermore, with the pulled safety syringe covered, the user needs not to feel concern about prick on the skin.

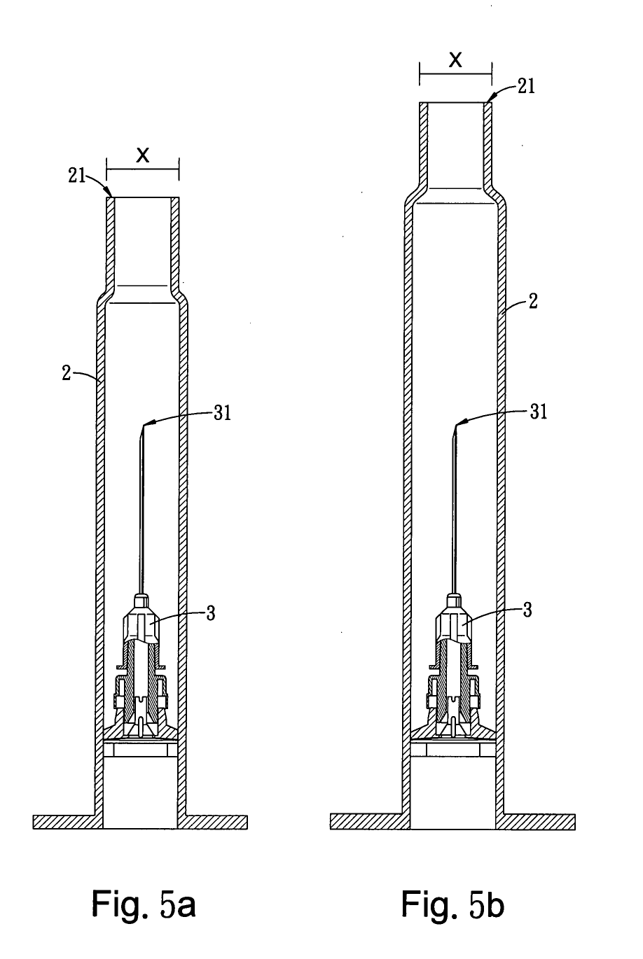

Referring to FIGS. 5a and 5b simultaneously, which show an embodiment of a structure of the syringe cover including the barrel, wherein the diameters X at the ends 21 of the hollow barrels 2 in two figures are identical, and thereby the structure can be implemented to the hollow barrel 2 with different capacity, especially a hollow syringe 2 with the capacity of 3 cc, 5 cc, and 10 cc. According to this safe design of protecting medical personnel from prick, the infection derived from needles can be radically eradicated.

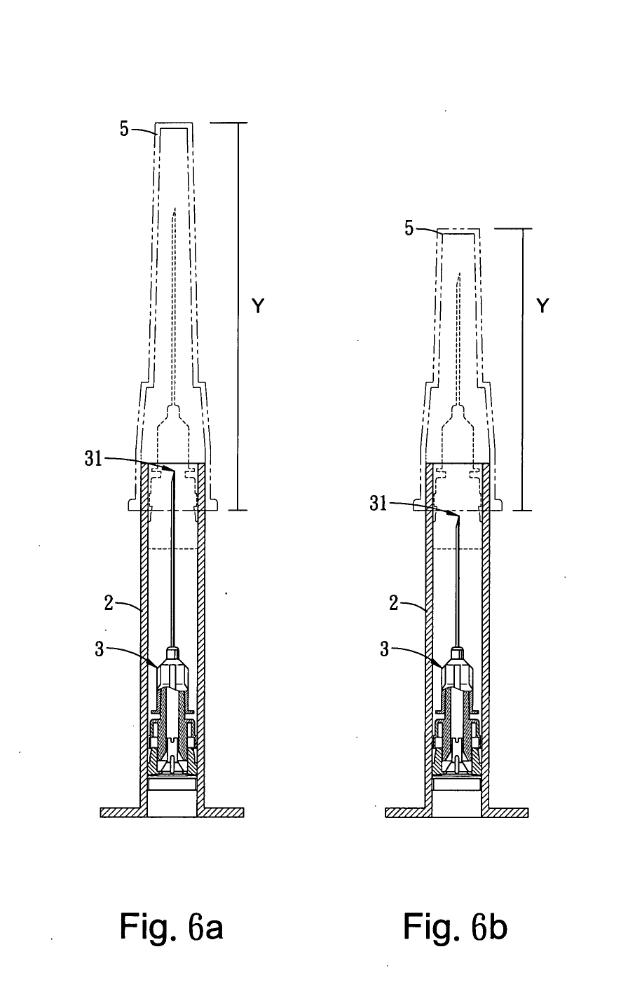

Referring to FIGS. 6a and 6b, which show an embodiment of a structure of the syringe cover including the barrel, wherein the length Y of the syringe cover 5 is designed to meet various standards (various lengths) of needle 3 according to specifications (lengths) of needle 3.

In light of the abovementioned descriptions, it is of course to be understood that the embodiment described herein is merely illustrative of the principles of the invention but not restricted to ambits for implementation of the invention. Furthermore, a wide variety of modifications thereto may be effected by persons skilled in the art without departing from the spirit and scope of the invention as set forth in the following claims.

What is claimed is:

1. A structure of the syringe cover including the barrel, which provides an implementation in the structure of the self-destruction syringe and the safety syringe with a capacity from 0.3 cc to 60 cc, and thereby accommodates syringes of differing capacity, is primarily characterized in that: the outer diameter at the aperture end of the barrel is identical to the inner diameter of the syringe cover, which is coupled to the aperture end of the barrel and then forms a hermetically sealed status of the whole syringe against danger of exposure of needles by mutual jostle between used syringes in medical wastes.

2. The structure of the syringe cover including the barrel according to claim 1, wherein the syringe cover assumes a conical form, and its inner wall indicates an inclined guide face.

3. The structure of the syringe cover including the barrel according to claim 1, wherein the structure at the aperture end of the barrel is implemented to a syringe with a capacity of 3 cc.

4. The structure of the syringe cover including the barrel according to claim 1, wherein the structure at the aperture end of the barrel is implemented to a syringe with a capacity of 5 cc.

5. The structure of the syringe cover including the barrel according to claim 1, wherein the structure at the aperture end of the barrel is implemented to a syringe with a capacity of 10 cc.
